(19) United States
(12) Patent Application Publication
(10) Pub. No.: US 2025/0327012 A1
(43) Pub. Date: Oct. 23, 2025

(54) A multi-well graphene-multielectrode array device for in vitro 3d electrical stimulation and method to obtain the device

(71) Applicants: UNIVERSIDADE DE AVEIRO, AVEIRO (PT); GRAPHENEST, S.A., Paradela SVV (PT); INSTITUTO DE TELECOMUNICAÇÕES, AVEIRO (PT)

(72) Inventors: Paula Alexandrina DE AGUIAR PEREIRA MARQUES, CACIA (PT); Luis Filipe MESQUITA NERO MOREIRA ALVES, MAIA (PT); Natália BRAZ BARROCA, AVEIRO (PT); Patrícia Alexandra ALMEIDA DIAS MARTINS, AVEIRO (PT); Bruno REIS FIGUEIREDO, BUSTOS (PT); Rui Pedro FONSECA FERREIRA DA SILVA, MACINHATA DO VOUGA (PT); Adriana Filipe BERNARDES, LEIRIA (PT); Vitor Emanuel MARQUES ABRANTES, AVEIRO (PT)

(73) Assignees: UNIVERSIDADE DE AVEIRO, AVEIRO (PT); GRAPHENEST, S.A., Paradela SVV (PT); INSTITUTO DE TELECOMUNICAÇÕES, AVEIRO (PT)

(21) Appl. No.: 18/861,386

(22) PCT Filed: Apr. 28, 2023
(86) PCT No.: PCT/IB2023/054462

§ 371 Date: Oct. 29, 2024

(30) Foreign Application Priority Data
Apr. 29, 2022 (PT) 117948

(51) Int. Cl. C12M 1/42; C12M 1/00; C12M 1/12; C12M 1/32

(52) U.S. Cl. CPC C12M 35/02; C12M 23/12; C12M 23/20; C12M 23/38; C12M 25/08; C12M 37/04

(57) ABSTRACT

A cell and tissue culture device and method to obtain same are disclosed. An embodiment includes a support electrode having a first electrically-non-conductive substrate sheet, a first patterned circuit made of a conductive ink applied on the substrate, a first plurality of patterned graphene dots connected to the first electrically conductive patterned circuit, a dielectric ink coating having patterned openings for exposing the graphene dots; a multi-well plate having a plurality of wells that receive the culture; a lid electrode having a second non-conductive substrate sheet; a second patterned circuit made of a electrically conductive ink applied on the substrate, a plurality of inserts; a second plurality of patterned graphene dots connected to the second electrically conductive patterned circuit. Each graphene dot is arranged on an insert to apply electrical stimulus to the culture and tissue, wherein the multi-well plate is between the support electrode-and the lid electrode.

### TECHNICAL FIELD

The present disclosure relates to a device for cell growth, proliferation and for tissue cultures by engineering, where simultaneous cues, such as biological, chemical, and electrical can be assessed. Particularly, it relates to a multi-well (or multiwell) graphene-multielectrode array (G-MEA) device for in vitro 3D electrical stimulation of cell lines and tissues and a method to obtain the said device.

### BACKGROUND

The design of a high-throughput multielectrode array (MEA) culture plate is highly desirable for the growth of cell cultures and tissue engineering.

The development of a high throughput culture plate needs to ensure a higher yield of widely distributed micro-scale electrodes and the manufacturing costs must be considered.

Most of the current and well-established MEA for 3D in vitro stimulation are spike or needle-like and possess low sensitivity, which does not completely mimic an in vivo environment.

The majority are based on metals being typically rigid and limited to what concerns electrical, mechanical, and biological cues (passivation and surface texturing to reduce impedance and enhance tissue integration are typically needed).

The document WO2015012955 discloses an electrophysiology culture plate formed from a MEA plate. The plate was formed by a combination of five conventional PCB processes: photoengraving, milling, etching, plating, and lamination. It discloses a substrate that has a plurality of vias, being in electrical contact with each of a plurality of contact pads disposed on the bottom of the device, allowing the connection to a controlling unit to perform electrical stimulation and recording. Despite the different PCB processing layers, document WO2015012955 discloses a MEA plate that allows the electrical stimulation of cell culture in a 2D manner/topography, requiring a working electrode, a counter electrode, and a reference electrode on the same quota. It discloses a MEA plate that has gold electrodes that are prepared by electroplating and micromachined with laser processes to increase the surface area and to have superior impedance performance. It discloses a MEA plate that is sterilized to alleviate any issues with cytotoxicity across multiple cell lines due to the potential leaching of copper and nickel used during the PCB processes, such as laser micromachining.

The document US 2020/0270561 A1 discloses a mechanical stimulator system that can maintain the sterility of biological samples within a multi-well plate while performing mechanical stimulation. The system can be configured to mimic real-world biological stimulations, such as the impacts on joints caused by walking or running. For example, the pistons can be reciprocated at about 1 Hz to simulate slow walking. It discloses a plurality of pistons configured to fit into guides and reciprocate within the guides to provide mechanical stimulation to the biological samples in the wells. Also discloses a mechanical stimulator system that comprises a box or other housing configured to include a temperature-controlled water flux and a gas sensor configured to control gas concentration levels in the wells.

The document US2010120626 discloses an apparatus and methods relating to the instrumentation development for high throughput network electrophysiology and cellular analysis. More specifically, provided herein are multi-well microelectrode arrays (MEAs) and methods for the development of such an apparatus in an inexpensive fashion with a flexible, ANSI/SBS-compliant (American National Standards Institute/Society for Biomolecular Screening) format. The microelectrode arrays (500 micrometres or smaller) are a grid metal (titanium, chromium, gold, platinum, silver, tin oxide, etc) microelectrodes tightly spaced (1 mm apart or smaller) useful for electrically stimulating and sensing/recording singular or network active cells, and tissue. The techniques described herein relate to the use of microfabrication in combination with certain large-area processes that have been employed to achieve multi-well MEAs in ANSI/SBS-compliant culture well formats, which are also transparent for inverted/backside microscopy compatibility

None of the previous discloses presents a 3-dimensional (3D) graphene-multielectrode array multi-well test platform that stimulates cells and cell tissues.

These facts are disclosed in order to illustrate the technical problem addressed by the present disclosure.

### GENERAL DESCRIPTION

The present disclosure relates to a universal non-invasive 3-dimensional (3D) graphene-multielectrode array (G-MEA) multi-well test platform.

Some of the advantages of the disclosure are:
the G-MEA is produced by conventional printing techniques, automatic or semi-automatic, such as screen-printing, pad-printing, gravure, flexography, offset, or similar, as well as in-mould labelling techniques, including injection moulding, compression moulding, blow moulding and thermoforming, or similar;
the G-MEA only allows to perform electrical stimulation and recording;
it is a non-invasive electrical stimulation using two electrodes (positive and negative) on different quotas allowing a tridimensionality (3D) on the electrical field applied, and thus the possibility to electrically stimulate not only cell lines and but also tissues;
use of graphene ink that does not require secondary treatments to enhance impedance;
G-MEA that does not expose any metal and generate any cytotoxicity across multiple cell lines;
the disclosure uses a direct current (DC) or an alternating current (AC);
the disclosure uses electrical stimulation at frequencies higher than 1 Hz;
it comprises the use of guides to guide electrodes attached to inserts in the wells;
discloses the use of an electrical stimulator system that comprises an electronic controlling unit;
allows the simultaneous measurement of different cues, such as chemical, biological, biochemical and electrophysiological;
allows to electrically stimulate three-dimensional singular or network active cells and tissue such as cardiac, neural, and muscular;
allows fluorescent microscopy and cell counting;
allows recording of three-dimensional network active cells and tissue.

The present disclosure relates to a cell and tissue culture device comprising:
a support electrode comprising:
   a first electrically non-conductive substrate sheet,
   a first patterned circuit layer made of an electrically conductive ink applied on the substrate,
   a first plurality of patterned graphene dots connected to the first electrically conductive patterned circuit, for applying electrical stimulus to the culture and tissue,
   a dielectric ink coating having patterned openings for exposing the graphene dots;
a multi-well plate comprising a plurality of wells for receiving the culture;
a lid electrode comprising:
   a second electrically non-conductive substrate sheet,
   a second patterned circuit made of an electrically conductive ink applied on the substrate,
   a plurality of inserts, each insert for inserting in a well,
   a second plurality of patterned graphene dots connected to the second electrically conductive patterned circuit, wherein each graphene dot is arranged on an insert for applying electrical stimulus to the culture and tissue;
wherein the multi-well plate is arranged between the support electrode and the lid electrode.

In an embodiment, the device comprises another lid, preferably a second lid, having a peripheral edge of the culture multi-well plate and an individual well cap for each well.

In an embodiment, the electrode lid comprises a further dielectric ink layer for protecting the patterned circuit.

In an embodiment, each graphene dot is arranged on an end of each insert.

In an embodiment, the support electrode is closing the bottom of the well.

The electrodes of the well and the electrodes of the insert form an electrical field between the first and the second electrodes, promoting the growth of cells and tissue cells of the culture.

In an embodiment, the support electrode is a positive electrode or a negative electrode.

In an embodiment, the lid electrode is a positive electrode or a negative electrode.

In an embodiment, the number of graphene dots on the lid electrode is equal to the number of the graphene dots on the support electrode.

In an embodiment, the number of graphene dots on the lid electrode lid is different from the number of the graphene dots on the support electrode, preferably the number of graphene dots on the lid electrode is inferior than the number of graphene dots on the support electrode.

In an embodiment, each well of the multiplate has the same electrical stimulus or each well has a different electrical stimulus.

In an embodiment, the graphene dots are made of graphene ink.

In an embodiment, the graphene ink is a biocompatible graphene ink. Preferably, the graphene ink is made of a green organic solvent, a polymeric binder and graphene nanoparticles.

In an embodiment, the ink of the patterned circuit layer is made of silver or copper or nickel-copper or mixtures thereof. Preferably is made of silver because it has good high electrical and thermal conductivity, chemical stability, relatively low cost, and its oxide form can conduct electrical signals as well. Silver inks also have excellent adhesion to flexible substrates.

In an embodiment, the substrate sheet is a polymeric sheet or a glass sheet. In particular, the substrate of the lid electrode is a polymeric sheet, preferably a flexible polymeric sheet for shaping the substrate.

In an embodiment, the polymeric sheet has a thickness of at least 50 micrometres.

In an embodiment, the substrate is a sheet of polyethylene terephthalate (PET) or polyethylene naphthalate (PEN) or polycarbonate (PC) or polyimide (PI) or polyvynil chloride (PVC).

In an embodiment, the multi-well plate and the inserts are made of polystyrene (PS), or polycarbonate (PC), or polyethylene terephthalate glycol (PETG) or polylactic acid (PLA). Preferably, the used polymers allow chemical or thermal or radiation sterilization. Preferably, the radiation is UV, gamma and e-beam sterilization.

In an embodiment, the support electrode comprises a first adhesive for attaching the support electrode to the multi-well plate.

In an embodiment, the lid electrode comprises a second adhesive for attaching the graphene dots to the insert.

Preferably the first and second adhesives are double-sided.

In an embodiment, the device comprises a further lid, a second lid to maintain sterility of the culture in the wells.

In an embodiment, the current is a direct current (DC) or an alternating current (AC).

In an embodiment, the device is obtainable by printing techniques and in-mould labelling techniques. The printing is selected from screen-printing, pad-printing, gravure, flexography or offset, among others. The in-mould labelling is selected from injection moulding, compression moulding, blow moulding or thermoforming, among others.

Preferably the device is obtainable by screen-printing.

The present disclosure also refers to a method to obtain the cell and tissue culture device comprising the steps of:
preparing the support electrode by
   cleaning the first electrically non-conductive substrate sheet with ethanol or other sterilizing solvent;
   printing on the first electrically non-conductive substrate sheet the first patterned circuit made of an electrically conductive ink;
   printing the first plurality of patterned graphene dots on top of the circuit layer; printing the dielectric ink coating on top of the plurality of patterned graphene dots and patterned circuit, having patterned openings for exposing the graphene dots;
applying a first adhesive on the electrode base, particularly on the dielectric ink coating;
attaching the supporting electrode to the multi-well plate;
preparing the lid electrode by
   cleaning the second electrically non-conductive substrate sheet with ethanol or other sterilizing solvent;
   printing on the second electrically non-conductive substrate sheet the second patterned circuit made of an electrically conductive ink;
   printing the second plurality of patterned graphene dots on top of the circuit layer;
   applying a second adhesive and attaching to the end of the inserts; attaching the electrode base to the multi-well plate;
connecting the lid electrode lid and the support electrode to a controlling unit.

In an embodiment, the method further comprises the step of printing the further dielectric ink coating around the patterned circuit on the lid electrode.

### BRIEF DESCRIPTION OF THE DRAWINGS

The following figures provide preferred embodiments for illustrating the disclosure and should not be seen as limiting the scope of the invention.

FIG. 1: Schematic representation of an embodiment of the support electrode and the culture wells plate, namely a top view of a 48 wells support electrode layout consisting in: 3 screen-printed overlapping layers where 1 is a polymeric sheet used as substrate; 2 is the silver tracks layer; 3 is the graphene dots layer that will be in contact with cells that are inside the wells; and 4 is the dielectric layer; and 6 is the 24-well no-bottom plate.

FIG. 2: Schematic representation of a side view of an embodiment of the multi-well graphene-multielectrode array device, where 1 is the substrate; 2 is the silver track layer; 3 is the graphene dots layer; 4 is the dielectric layer; 5 is the adhesive; and 6 are the no-bottom wells.

FIG. 3: Schematic representation of an embodiment of the lid electrode, particularly a top view, consisting of: 3 screen-printed overlapping layers where 1 is a polymeric sheet used as substrate; 2 is the silver tracks layer; and 3 is the graphene dots layer that will be in contact with cells that are inside the wells 6; 4 is the dielectric layer and the 7 is the inserts.

FIG. 4: Schematic representation of the side view of an embodiment of the lid electrode, preferably the electrode layout and a lid where: 1 is a polymeric sheet used as substrate; 2 is the silver tracks layer; 3 is the graphene dots layer that will be in contact with cells that are inside the wells; 4 is the dielectric layer; 5 is the double-sided adhesive; and 7 is the inserts.

FIG. 5: Schematic representation of an embodiment of the device, namely a side view of the support electrode and lid electrode where: 1 is a polymeric sheet used as substrate; 2 is the silver tracks layer; 3 is the graphene dots layer that will be in contact with cells that are inside the wells; and 4 is the dielectric layer; 5 is the doubled-sided adhesive; 6 is the no-bottom wells; and 7 is the inserts.

FIG. 6: Schematic representation of an embodiment of a stimulation device comprising: 2 units of 24-wells no-bottom plates (48 wells) support electrode and 2 out 3 (32 wells) lid, preferably lid electrodes, for allowing the stimulation of 32 channels.

FIG. 7: Graphic representation of the electrochemical impedance spectroscopy (EIS) results show data at frequencies above 1 Hz, but it provides useful information for the nearby region.

### DETAILED DESCRIPTION

The present disclosure relates to a cell and tissue culture device comprising:
a support electrode comprising:
   a first electrically non-conductive substrate sheet;
   a first patterned circuit layer made of an electrically conductive ink applied on the substrate,
   a first plurality of patterned graphene connected to the first electrically conductive patterned circuit, for applying electrical stimulus to the culture and tissue,
   a dielectric ink coating having patterned openings for exposing the graphene dots;
a multi-well plate comprising a plurality of wells for receiving the culture;
a lid electrode comprising:
   a second electrically non-conductive substrate sheet;
   a second patterned circuit made of an electrically conductive ink applied on the substrate,
   a plurality of inserts, each insert for inserting in a well,
   a second plurality of patterned graphene dots connected to the second electrically conductive patterned circuit, wherein each graphene dot is arranged on an insert for applying electrical stimulus to the culture and tissue,
wherein the multi-well plate is arranged between the support electrode and the lid electrode.

Preferably, the graphene dots are exclusively in contact with cells that are inside the wells.

In an embodiment, the device is obtainable by screen-printing and in-mould labelling and comprises:
a support electrode comprising:
   a first electrically non-conductive substrate made of a polymeric sheet,
   a first patterned circuit layer made of an electrically conductive ink applied on the substrate,
   a first plurality of patterned graphene dots connected to the electrically conductive patterned circuit, for applying electrical stimulus to the culture and tissue,
   a dielectric ink coating having patterned openings for exposing the graphene dots,
a plate comprising a plurality of no-bottom wells for receiving the cell culture and tissue, wherein the support electrode closes a bottom of each well;
a lid electrode comprising:
   a second electrically non-conductive substrate sheet,
   a second patterned circuit made of an electrically conductive ink applied on the substrate,
   a second plurality of patterned graphene dots connected to the second electrically conductive patterned circuit, wherein each graphene dot is arranged on an insert for applying electrical stimulus to the culture and tissue,
   a further dielectric ink layer, for protecting the patterned circuit,
wherein the multi-well plate is arranged between the support electrode and the lid electrode.

In an embodiment, each end of the insert comprises an electrode for applying electrical stimulus to the cell culture and the tissue made by a graphene layer printed on top of a patterned circuit made of electrically conductive ink.

In an embodiment, a second lid comprising a peripheral edge of the culture plate and an individual well cap for each culture well.

In an embodiment, the support electrode is attached to the bottom of the culture wells plate by means of a double-sided adhesive tape laser cut with the exact same design of the dielectric layer, preferably dielectric ink to expose the first plurality of patterned graphene dots.

In an embodiment, the lid electrode, preferably the second plurality of patterned graphene dots, is attached to the end of the inserts of the lid by means of a double-sided adhesive, preferably a tape adhesive.

In an embodiment, the support electrode is a positive electrode or a negative electrode and the lid electrode is a positive electrode or a negative electrode.

In an embodiment, the number of the negative electrodes may not be the same as the number of wells in the plate.

In an embodiment, the lid electrode has guides to align and guide the electrodes along a vertical path of motion relative to the wells facilitating the inflow and outflow of media to and from the well during the electrical stimulation.

In an embodiment, the positive and negative electrodes can be swapped during the electrical stimulation according to the direction of the current flow.

In an embodiment, a second lid provides a shield to maintain the sterility of the biological samples in the wells.

In an embodiment, the method to obtain the cell culture and the tissue device described previously, is by printing (preferably screen-printing) and in-mould labelling and comprises the steps of:
Clean the polymeric substrate with ethanol or another sterilizing solvent;
Print on the polymeric substrate the patterned circuit layer made of an electrically conductive ink;
Print the patterned graphene layer on top of the patterned circuit layer;
Print the dielectric ink layer on top of the graphene layer and circuit layer, having specific patterns to expose the graphene dots;
Attach one of the sides of the double-sided adhesive on top of the dielectric layer;
Attach the non-bottom wells plate (or the inserts first lid) to the other side of the double-sided adhesive to finish the positive (or negative) electrode;
Connect the electrodes to an electronic controlling unit.

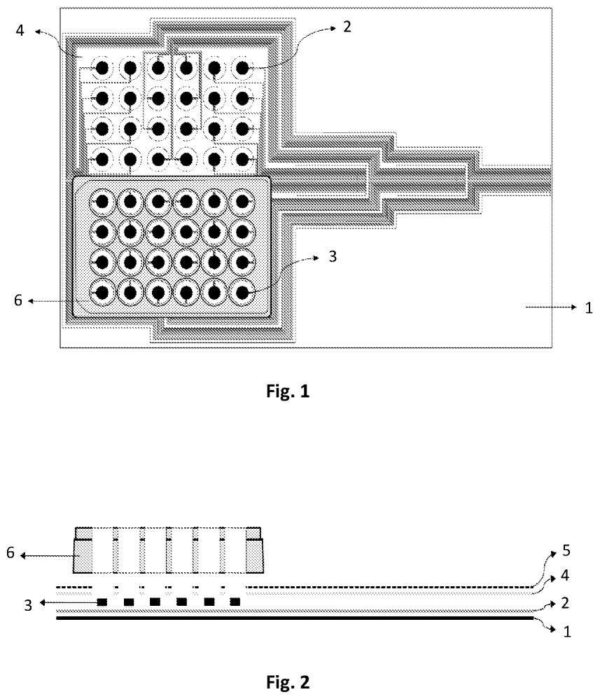

In an embodiment, FIGS. 1 and 2 are a schematic representation of an embodiment of the support electrode layout plus the culture wells, namely a top view and a side view of a 48 wells support electrode layout consisting in: 3 screen-printed overlapping layers where 1 is a polymeric sheet used as substrate; 2 is the first patterned circuit, preferably silver tracks layer; 3 is the plurality of patterned graphene dots, preferably graphene dots layer, that will be in contact with cells that are inside the wells; and the dielectric layer 4. It is also represented the double-sided adhesive 5 and the 24-well no-bottom plate 6.

In an embodiment, the wells do not have a bottom and the support electrode closes the bottoms of the wells. In an embodiment, the plate 6 comprises 48 wells, and each well can have different stimulus or groups of different stimuli.

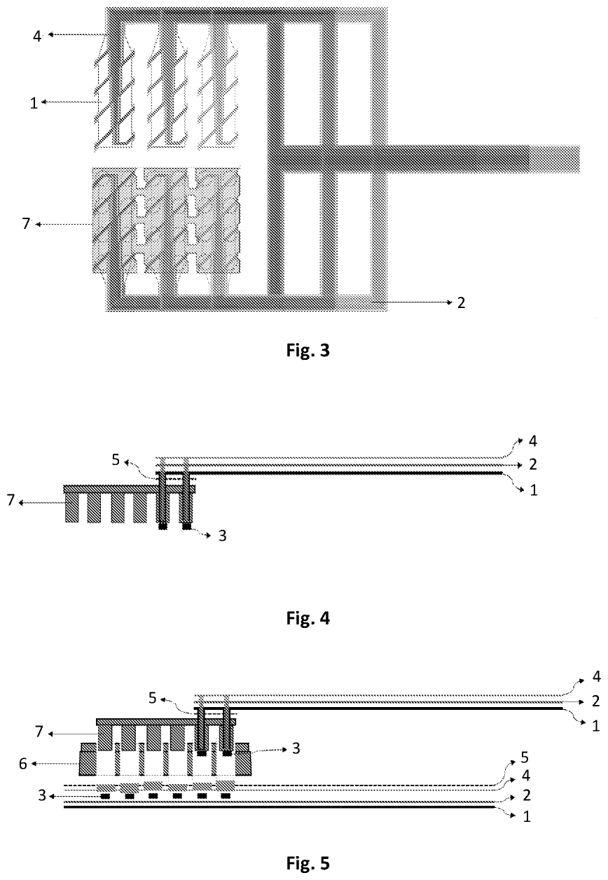

In an embodiment, FIGS. 3 and 4 are a schematic representation of an embodiment of the lid electrode layout, particularly a top view and side view of the lid electrodes layout of the device consisting in: 3 screen-printed overlapping layers where 1 is a polymeric sheet used as substrate; 2 is the second patterned circuit, preferably the silver tracks layers; 3 is the graphene dots layer that will be in contact with cells that are inside the wells; and the dielectric ink layer 4. It is also illustrated the double-sided adhesive 5 and the inserts 7.

In an embodiment, FIG. 5 is a schematic representation of an embodiment of the device, namely a side view of the support and lid electrodes comprising in: the electrodes where 1 is a polymeric sheet used as substrate; 2 is the patterned circuits, preferably is the silver tracks layer; 3 is the graphene dots layer that will be in contact with cells that are inside the wells; and the dielectric layer 4; the double-sided adhesive 5; and the no-bottom wells 6 and inserts 7.

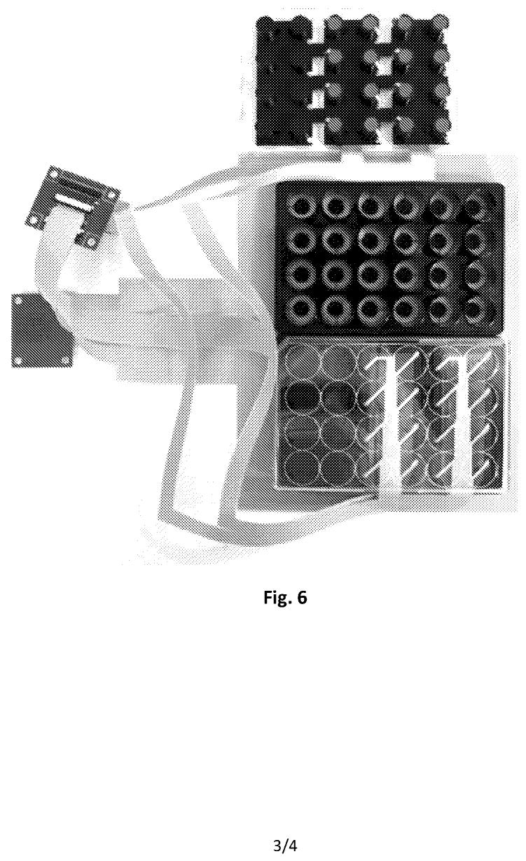

In an embodiment, FIG. 6 is a schematic representation of an embodiment of a stimulation device comprising: the support electrode with 48 wells; 2 units of 24- of well no-bottom plates; and 2 out 3 lid electrodes, allowing the stimulation of 32 channels.

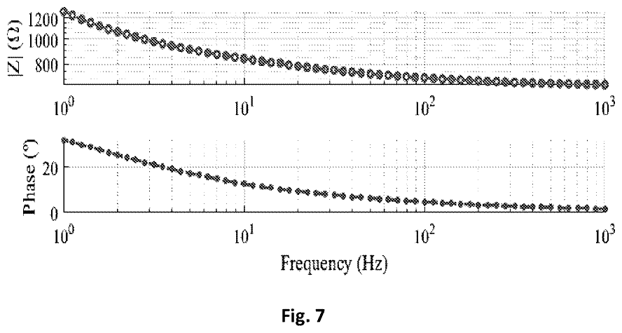

In an embodiment, FIG. 7 represents an electrochemical impedance spectroscopy (EIS) results show the electrode characteristics (i.e., the overall impedance of device at operating at frequencies higher than 1 Hz). This example represents the overall impedance of the device plus a commercial culture medium-Minimum Essential Medium Eagle—as a function of the frequency in the range of 1-1000 Hz (AC current stimulus). The overall impedance renders values below 1300 Ω on the entire frequency range.

In an embodiment, the substrate sheet can be selected from a polymeric sheet or a glass sheet. Preferably, the lid electrode comprises a polymeric sheet and more preferably a flexible sheet.

In an embodiment, the substrate is transparent in the vicinity of the microelectrodes.

In an embodiment, the electrode lid is flexible. The flexibility of the polymeric sheet allows the definition of geometry. The support electrode can comprise a polymeric sheet or a glass sheet.

In an embodiment, the substrate is Polyethylene terephthalate (PET) sheets, preferably with thickness higher than 50 micrometres. Polyethylene naphthalate (PEN) or polycarbonate (PC) or polyimide (PI) or polyvynil chloride (PVC) sheets can also be used.

In an embodiment, the lid electrode and the support electrode are screen- printed using automatic or semi-automatic equipment and built by in-mould labelling.

In an embodiment, the device and a culture medium provide an overall impedance below 1300 Ohm at frequencies higher than 1 Hz.

In an embodiment, each device uses 2 units of a plate with 24 wells with no bottom made of polystyrene (PS).

In an embodiment, each device uses 2 units of a lid electrode with 24 inserts made of Polyethylene terephthalate glycol (PETG).

In an embodiment, each device uses 2 units of a second lid made of polycarbonate (PC).

In an embodiment, preferably the device comprises 48 electrodes and 48 wells.

In an embodiment, 16 wells have the same stimuli.

In an embodiment, the stimulus is made for 48 wells, where every 16 wells have the same one type of stimulus.

In an embodiment, the multi-well plate can comprise 1, 6, 12, 24, 48, 96, 384 and 768 culture wells.

In an embodiment, the device comprises a biocompatible graphene ink for the graphene dots.

Preferably, the properties of the graphene ink are:

| | |
|---|---|
| Solids Content by Weight (%) | 14 |
| Viscosity @shear rate of 10 s⁻¹ (mPa · s) | 2700-5200 |
| Sheet Resistivity @ 20 μm film thickness (ohms/sq) | 1000 |

In an embodiment, the device comprises silver ink as a patterned circuit. Preferably, the properties of this ink are:

| | |
|---|---|
| Solids Content by Weight (%) | 100 |
| Density (kg/m³) | 2672.13 |
| Viscosity, Brookfield CP42, 25° C., Speed 50 rpm (mPa · s) | 800 |
| Theoretical Coverage @ 25 μm (m²/kg) | 3.84 |
| Sheet Resistivity @ 25 μm film thickness (ohms/sq) | 0.006 |

In an embodiment, the device comprises a dielectric ink. Preferably the properties of this ink are:

| | |
|---|---|
| Viscosity, Haake RS1 C20/2° TiL at 230 sec−1 at 25° C. | 5500-8000 |
| (mPa · s) | |
| Theoretical coverage using 230 mesh stainless steel screen | 80 |
| (m²/kg) | |

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities for modifications thereof. The above described embodiments are combinable.

The following claims further set out particular embodiments of the disclosure.

1. A cell and tissue culture graphene-multielectrode array device, comprising:
a support electrode, comprising:
a first electrically non-conductive substrate sheet,
a first patterned circuit made of an electrically conductive ink applied on the substrate,
a first plurality of patterned graphene dots connected to the first electrically conductive patterned circuit, for applying electrical stimulus to the culture and tissue, and
a dielectric ink coating having patterned openings for exposing the graphene dots;
a multi-well plate comprising a plurality of wells for receiving the culture;
a lid electrode, comprising:
a second electrically non-conductive substrate sheet,
a second patterned circuit made of an electrically conductive ink applied on the substrate,
a plurality of inserts, each insert for inserting in a well, and
a second plurality of patterned graphene dots connected to the second electrically conductive patterned circuit, wherein each graphene dot is arranged on an insert for applying electrical stimulus to the culture and tissue;
wherein the multi-well plate is arranged between the support electrode and the lid electrode.

2. The device according to claim 1, wherein the lid electrode comprises a further dielectric ink.

3. The device according to claim 1, wherein each graphene dot is arranged on an end of each insert.

4. The device according to claim 1, wherein the support electrode closes the bottom of the well.

5. The device according to claim 1, wherein the support electrode is a positive electrode or a negative electrode.

6. The device according to claim 1, wherein the lid electrode is a negative electrode or a positive electrode.

7. The device according to claim 1, wherein the graphene dots are made of graphene ink.

8. The device according to claim 7, +wherein the graphene ink is a biocompatible graphene ink.

9. The device according to claim 1, wherein the patterned circuit is made of silver ink or copper ink or nickel-copper ink or their mixture.

10. The device according to claim 1, wherein the substrate sheet is a polymeric or a glass sheet.

11. The device according to claim 1, wherein the substrate sheet is a polymeric sheet.

12. The device according to claim 1, wherein the polymeric sheet has a thickness of at least 50 micrometres.

13. The device according to claim 10, wherein the substrate is a sheet of polyethylene terephthalate or polyethylene naphthalate or polycarbonate or polyimide or polyvynil chloride.

14. The device according to claim 1, wherein the multi-well plate and the inserts are made of polystyrene, or polycarbonate, or polyethylene terephthalate glycol or polylactic acid.

15. The device according to claim 1, wherein the support electrode comprises a first adhesive for attaching the support electrode to the multi-well plate.

16. The device according to claim 1, wherein the lid electrode comprises a second adhesive for attaching the graphene dots to the insert.

17. The device according to claim 1, further comprising a lid to maintain the sterility of the culture in the wells.

18. The device according to claim 1, wherein said device is a printed device.

19. The device according to claim 1, wherein said device is an in-mould labelled device.

20. The device according to claim 18, wherein the printed device is made by screen-printing.

21. A method for obtaining the cell and tissue culture graphene-multielectrode array device according to claim 1, comprising the steps of
preparing the support electrode by:
cleaning the first electrically non-conductive substrate sheet with ethanol or other sterilizing solvent;
printing on the first electrically non-conductive substrate sheet the first patterned circuit made of an electrically conductive ink;
printing the first plurality of patterned graphene dots on top of the circuit layer; and
printing the dielectric ink coating around of the plurality of patterned graphene dots and patterned circuit, having patterned openings that allow the exposure of the graphene dots;
applying a first adhesive on the support electrode base, particularly on the dielectric ink coating;
preparing the lid electrode by:
cleaning the second electrically non-conductive substrate sheet with ethanol or other sterilizing solvent;
printing on the second electrically non-conductive substrate sheet the second patterned circuit made of an electrically conductive ink;
printing the second plurality of patterned graphene dots on top of the circuit layer, and
applying a second adhesive on the graphene dots for attaching to the end of the inserts;
attaching the electrode base to the multi-well plate; and
connecting the lid electrode and the support electrode to a controlling unit.

22. The method according to claim 21, further comprising the step of printing a further dielectric ink layer on the lid electrode.
